(19) United States
(12) Patent Application Publication
(10) Pub. No.: US 2010/0125931 A1
(43) Pub. Date: May 27, 2010

(54) ATHLETIC SCARF WITH HEAT PACK

(76) Inventor: Alan Arakelian, Sherman Oaks, CA (US)

Correspondence Address: GEORGE A LEONE, SR;CITADEL PATENT LAW, 9124 Gravelly Lake Drive SW, SUITE 102, Lakewood, WA, 98499, US

(21) Appl. No.: 12/560,286

(22) Filed: Sep. 15, 2009

Related U.S. Application Data
(63) Continuation-in-part of application No. 29/312,983, filed on Nov. 25, 2008, now abandoned.

(51) Int. Cl. A42B 5/00; A61F 7/00

(52) U.S. Cl. 2/207; 607/109

(57) ABSTRACT

An athletic scarf includes a flat, pliable, substantially rectangular body having a first end and a second end and a top end and a bottom end, and a facing surface and an opposing interior surface, bounded by the first and second ends and top and bottom edges. An inner strip is fastened to the interior surface to form a strip of material having a smaller area than the interior surface that runs coextensively with along the length of the interior surface and holds a removable heat pack. First and second ends of the rectangular body are joinable to form a tube sized to fit around a wearer's neck.

### RELATED APPLICATION

This application is a continuation-in-part of co-pending U.S. design application number 29/312,983 of Alan Arakelian, filed Nov. 25, 2008.

### TECHNICAL FIELD

The present invention relates to an article of wearing apparel, and more particularly to athletic scarves and the like.

### BACKGROUND

Neck apparel has long been used for protection from the elements and for decorative purposes. Some sports, such as U.S. style football, are played outdoors in frigid weather conditions. Some sports fans, especially in sports such as soccer, wear scarves that include team logos as a symbol of team loyalty. Until the present invention, no neck piece has been available that combines a warming ability for comfort and safety with a stylish display of team logos or other decorative emblems.

### BRIEF SUMMARY OF THE DISCLOSURE

An athletic scarf includes a flat, pliable, substantially rectangular body having a first end, a second end, a top end and a bottom end, and further having a facing surface and an opposing interior surface, bounded by the first and second ends and top and bottom edges. An inner strip is fastened to the interior surface to form a strip of material having a smaller area than the interior surface and runs coextensively along the length of the interior surface, wherein the inner strip includes at least one means for installing at least one removable heat pack. A first fastening means is attached to the interior surface proximate to its first end. A second fastening means is attached to the facing surface of the rectangular body proximate to a second opposite end, wherein the facing surface includes means for attaching an insignia patch. The first and second ends of the rectangular body are fastenable to each other at the first and second fastening means. The first and second fastening means are positioned such that when the first end is attached to the second end, the rectangular body forms a tube having inner and outer circumferential surfaces provided by the first and second opposing surfaces of the rectangular body. The tube is thereby sized to fit around a wearers neck.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 schematically shows an example of an exterior surface of an athletic scarf.

FIG. 2 schematically shows an example of an interior surface of an athletic scarf

FIG. 3 schematically shows an example of heat pack installation into an athletic scarf.

FIG. 4 schematically shows an example of a method for constructing an athletic scarf.

FIG. 5 schematically shows an example of an alternate embodiment of an athletic scarf.

FIG. 6 illustrates an example of an athletic scarf when fastened into a tube shape as when worn around a wearer's neck.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF THE PREFERRED EMBODIMENTS

The following disclosure describes several embodiments and methods for an athletic scarf with a heat pack. Several elements in accordance with example embodiments are set forth and described in the Figures. It will be appreciated that elements and methods in accordance with other example embodiments can include additional procedures or features different than those shown in the Figures. Example embodiments are described herein. However, it will be understood that these examples are for the purpose of illustrating the principles, and that the invention is not so limited. Additionally, elements and methods in accordance with several example embodiments may not include all of the features shown in the Figures.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or various combinations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

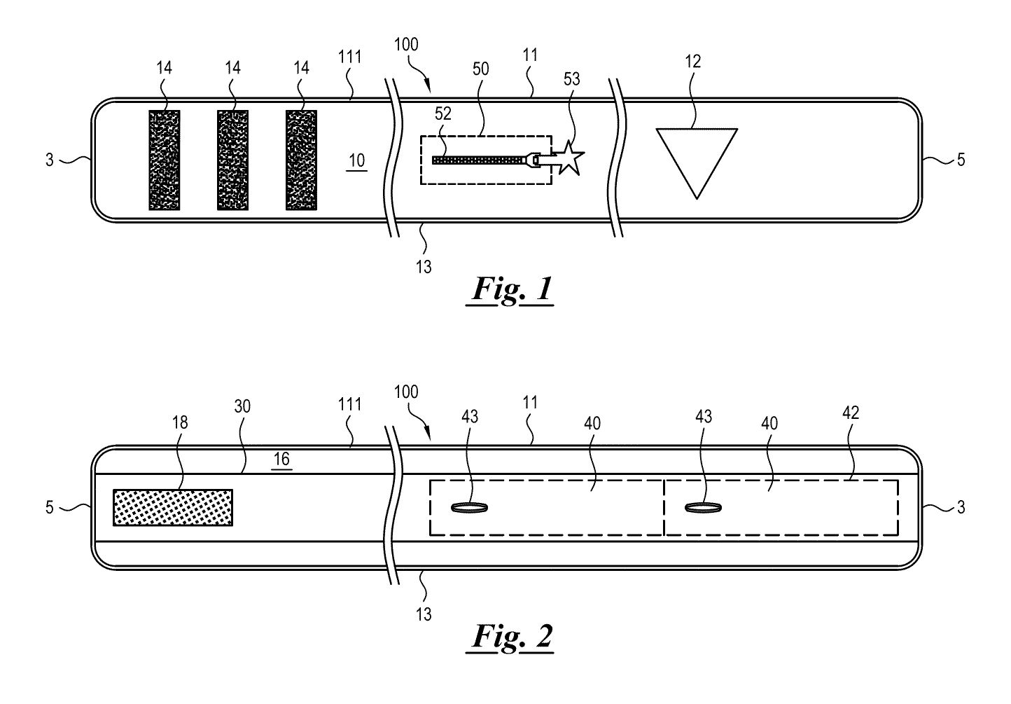

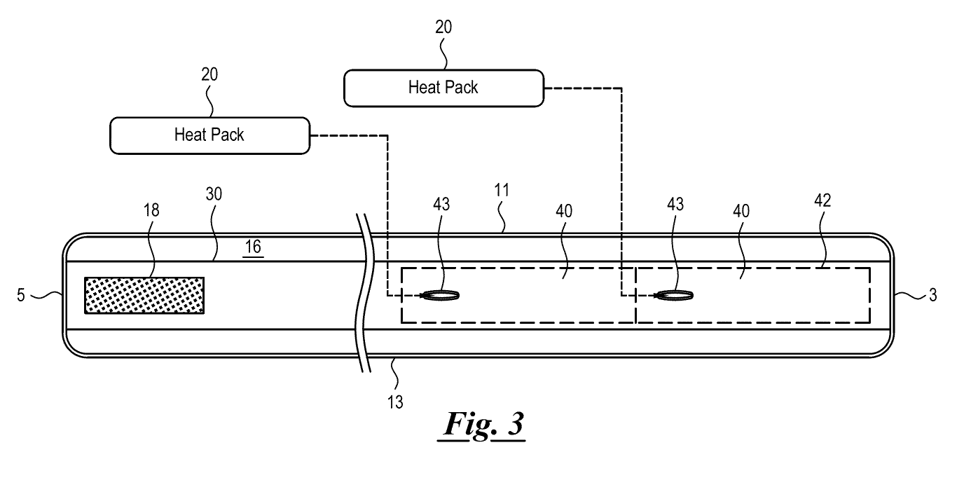

Referring now jointly to FIG. 1 and FIG. 2, an example of an athletic scarf is shown. The athletic scarf includes a flat, pliable, substantially rectangular body 100 having a first end 5, a second end 3, a top end 11 and a bottom end 13. A facing surface 10 and an opposing interior surface 16 are each bounded by the first and second ends and top and bottom edges. An inner strip 30 is fastened to the interior surface 16 to form a strip of material having a smaller area than the interior surface 16 and running coextensively with along the length of the interior surface 16. The inner strip 30 includes at least one means for installing at least one removable heat pack 20 (as shown in FIG. 3). A first fastening means 18 is attached to the interior surface 16 proximate to its first end 5. A second fastening means 14 is attached to the facing surface 10 of the rectangular body proximate to its second end 3, wherein the facing surface 10 includes a space for attaching an insignia patch 12 or other decorative element. The insignia patch 12 may be permanently affixed or removable so as to allow changing patches from time to time. A liner 42 may advantageously be attached, inserted or otherwise affixed between the inner strip 30 and the interior surface 16. The liner 42 may advantageously be a material selected from the group consisting of a plastic material, a latex material, a liquid resistant material or equivalents and combinations thereof. In some embodiments, the liner may not be required.

In one example embodiment the first end 5, the second end 3, the top end 11 and the bottom end 13 comprise a continuous hemmed edge 111. In one example, the first and second fastening means may advantageously be complementary pieces of hook-and-loop material. The rectangular body may advantageously be a material selected from the group comprising woven cotton, synthetic materials, wool, cloth, woven material, knitted material, yarn, nylon, polyester, elastic material, micro-fiber material, fleece or equivalents and combinations thereof. The inner strip 30 may advantageously be a material selected from the group comprising woven cotton, synthetic materials, nylon, polyester, elastic material, micro-fiber material, fleece or equivalents and combinations thereof. In a preferred embodiment, the first fastening means is selectably fastenable to one of the plurality of substantially parallel strips of hook-and-loop material, such as is commercially available under the brand name Velcro®, wherein the second fastening means comprises a plurality of substantially parallel strips of hook-and-loop material, thereby adjusting the size of the tube formed by the rectangular body.

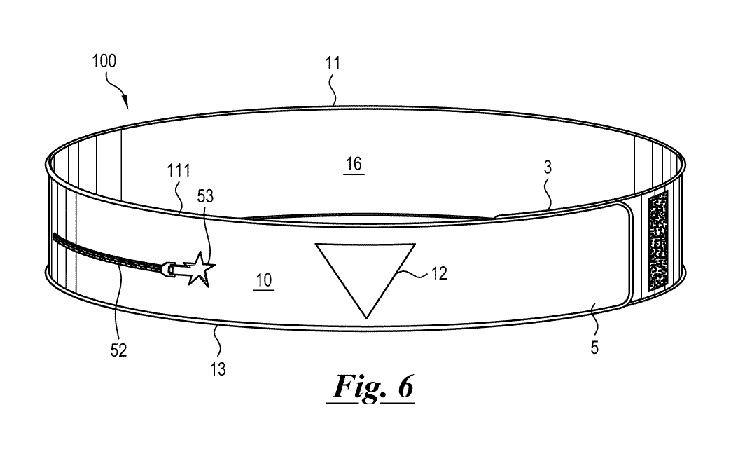

In another example, the at least one means for installing at least one removable heat pack includes a slot 43 having edges lined with joinable fasteners, such as, for example, hook and loop fasteners. The first and second ends of the rectangular body 100 may advantageously be joinable to each other at the first and second fastening means, the first and second fastening means being positioned such that when the first end is attached to the second end, the rectangular body forms a tube having inner and outer circumferential surfaces provided by the first and second opposing surfaces 10, 16 of the rectangular body, the tube being sized to fit around a wearer's neck as illustrated in FIG. 6. Further, the first and second fastening means 18, 14 comprise joinable means on the opposing surfaces 10, 16 for fastening the scarf about a wearer's neck, wherein the first fastening means is joinable at a plurality of locations along the second fastening means 14 so as to vary the circumference of the scarf to closely match the circumference of the wearer's neck.

In yet another example an optional pocket 50, accessible through a pocket zipper 52 on facing surface 10, may be added. A zipper pull 53 may have the shape of a charm or other decorative element. The pocket 50 may be sown onto the inner strip 30 or formed within inner strip 30. This optional feature adds a stylistic flair as well as providing extra storage in the athletic scarf, as for example, cash, lip balm or the like can be carried while walking, jogging or during other activities.

Referring now to FIG. 3, an example of heat pack installation into an athletic scarf is schematically shown. The at least one removable heat pack 20 is installed within the inner strip 30. In one example, the heat pack 20 comprises a heat pack selected from the group consisting of a gel pack, a reusable heat pack, a therapeutic heat pack, a non-electric heat pack, a sodium acetate solution heat pack, and an electric heat pack or equivalents. The at least one removable heat pack 20 may advantageously be installed into at least one compartment 40 formed within the liner 42, between the liner and the inner strip 30 or between the inner strip 30 and the interior surface 16. Other equivalent configurations may also be used.

One useful type of commercially available heat pack is a sodium acetate heat pad with a reusable heat reservoir. It typically contains a supersaturated solution of sodium acetate (NaCH3COO). Crystallization is triggered by flexing a small flat disc of notched ferrous metal embedded in the liquid. Pressing the disc releases adhered crystals of sodium acetate into the solution which then act as nucleation sites for the recrystallization of the remainder of the salt solution. Because the liquid is supersaturated, this makes the solution crystallize suddenly, thereby releasing the energy of the crystal lattice. The pad can be reused by placing it in boiling water for 10-15 minutes, which redissolves the sodium acetate in the contained water and recreates a supersaturated solution. Once the pad has returned to room temperature it can be triggered again.

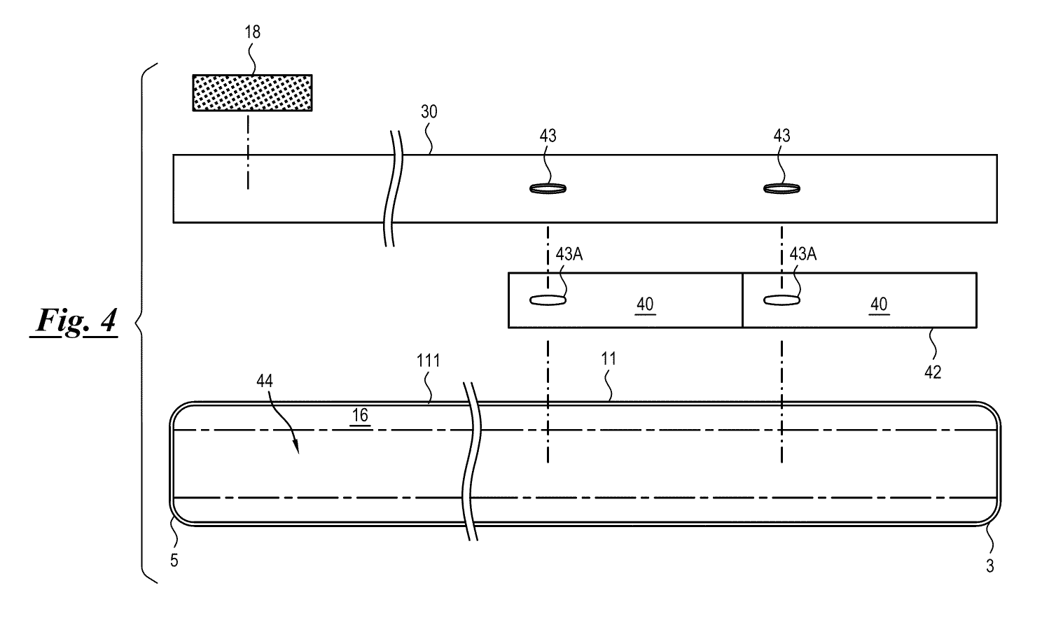

Referring now to FIG. 4, an exploded view of an example of a method for constructing an athletic scarf is schematically shown. A flat, pliable, substantially rectangular body 100 having a first end 5, a second end 3, a top end 11 and a bottom end 13, and further having a facing surface and an opposing interior surface 16, bounded by the first and second ends and top and bottom edges. The inner strip is fastened to the interior surface 16 to form a strip of material having a smaller area than the interior surface 16. The inner strip 30 runs coextensively along the length of the interior surface 16. At least one compartment 40 for installing at least one removable heat pack 20 is formed by hemming the inner strip around the liner 42. The liner 42 may advantageously be a latex liner. In one example the liner may comprise a pouch having a plurality of slots 43A where each slit opens to a compartment 40. Slots 43A are located to coincide with the closeable slots 43 in the inner strip 30. The first fastening means 18 is attached to the inner liner 30 proximate to the first end 5 within a central region 44. The at least one removable heat pack 20 is installed into at least one compartment 40. In one example, two heat packs may be installed into compartments 40 in the liner 42.

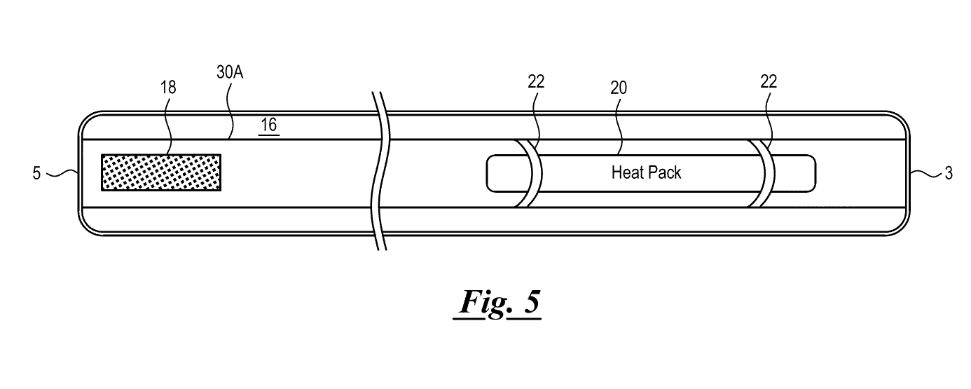

Referring now to FIG. 5, an example of an alternate embodiment of an athletic scarf is schematically shown. The alternate embodiment is configured and constructed essentially identically to the examples described above, except the at least one heat pack 20 is held externally to a modified inner liner 30A by straps 22. Further, the liner 42 may be eliminated from this configuration. The modified inner liner 30A need not include a compartment or openings for housing the at least one heat pack.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by specifically different equipment, and devices, and that various modifications, both as to the equipment details and operating procedures, may be accomplished without departing from the true spirit and scope of the present invention.

What is claimed is:

1. An athletic scarf, comprising:
a flat, pliable, substantially rectangular body having a first end, a second end, a top end and a bottom end, and further having a facing surface and an opposing interior surface, bounded by the first and second ends and top and bottom edges, wherein the facing surface includes means for attaching an insignia patch;
an inner strip fastened to the interior surface to form a strip of material having a smaller area than the interior surface, where the inner strip runs coextensively along the length of the interior surface, wherein the inner strip is joined with the interior surface to include at least one means for installing at least one removable heat pack;
first fastening means attached to the interior surface proximate to its first end;
second fastening means attached to the facing surface of the rectangular body proximate to a second end at a side opposite the first end; and
the first and second ends of the rectangular body being fastenable to each other at the first and second fastening means, the first and second fastening means being positioned such that when the first end is attached to the second end, the rectangular body forms a tube having inner and outer circumferential surfaces provided by the first and second opposing faces of the rectangular body, the tube being sized to fit around a wearer's neck.

2. The athletic scarf of claim 1 wherein the first end, second end, top end and bottom end comprise a continuous hemmed edge.

3. The athletic scarf of claim 1 wherein the first and second fastening means comprise joinable means on the opposing faces for fastening the scarf about the wearer's neck, wherein the first fastening means is joinable at a plurality of locations along the second fastening means so as to vary the circumference of the scarf to closely match the circumference of the wearers neck.

4. The athletic scarf of claim 1 further comprising at least one removable heat pack 20 installed within the inner strip.

5. The athletic scarf of claim 4 wherein the heat pack comprises a heat pack selected from the group consisting of a gel pack, a reusable heat pack, a therapeutic heat pack, a non-electric heat pack, a sodium acetate solution heat pack, and an electric heat pack.

6. The athletic scarf of claim 1 further comprising at least one removable heat pack installed into at least one compartment formed by the inner strip.

7. The athletic scarf of claim 6 wherein the at least one removable heat pack comprises a heat pack selected from the group consisting of a gel pack, a reusable heat pack, a therapeutic heat pack, a non-electric heat pack, a sodium acetate solution heat pack, and an electric heat pack.

8. The athletic scarf of claim 1, wherein the first and second fastening means comprise complementary pieces of hook-and-loop material.

9. The athletic scarf of claim 1, wherein the rectangular body comprises a material selected from the group comprising woven cotton, synthetic materials, wool, cloth, woven material, knitted material, yarn, nylon, polyester, elastic material, micro-fiber material, fleece and combinations thereof.

10. The athletic scarf of claim 1, wherein the inner strip comprises a material selected from the group comprising woven cotton, synthetic materials, wool, cloth, woven material, knitted material, yarn, nylon, polyester, elastic material, micro-fiber material, fleece and combinations thereof.

11. The athletic scarf of claim 1, further comprising a liner interposed between the inner strip and the interior surface.

12. The athletic scarf of claim 11, wherein the liner comprises a material selected from the group consisting of a plastic material, a latex material, a liquid resistant material and combinations thereof.

13. The athletic scarf of claim 1, wherein the second fastening means comprises a plurality of substantially parallel strips of hook-and-loop material, and wherein the first fastening means is selectably fastenable to one of the plurality of strips of hook-and-loop material.

14. The athletic scarf of claim 1 wherein the at least one means for installing at least one removable heat pack includes a slot having edges lined with joinable fasteners.

15. A method for making an athletic scarf, comprising:
forming a flat, pliable, substantially rectangular body having a first end, a second end, a top end and a bottom end, and further having a facing surface and an opposing interior surface, bounded by the first and second ends and top and bottom edges;
fastening an inner strip to the interior surface to form a strip of material having a smaller area than the interior surface and running coextensively with along the length of the interior surface;
forming a compartment within the inner strip for installing at least one removable heat pack;
attaching a first fastening means to the interior surface proximate to a first end;
attaching a second fastening means to the facing surface of the rectangular body proximate to a second end, wherein the facing surface includes means for attaching an insignia patch; and
positioning the first and second fastening means such that when the first end is attached to the second end, the rectangular body forms a tube having inner and outer circumferential surfaces provided by the first and second opposing surfaces of the rectangular body.

16. The method of claim 15 wherein the first and second fastening means comprise joinable means for fastening the rectangular body about a wearer's neck, wherein the first fastening means is joinable at a plurality of locations along the second fastening means so as to vary the circumference of the rectangular body to closely match the circumference of the wearer's neck.

17. The method of claim 15 further comprising installing the at least one removable heat pack into at least one compartment formed by the inner strip.

18. The method of claim 17 wherein the at least one heat pack comprises a heat pack selected from the group consisting of a gel pack, a reusable heat pack, a therapeutic heat pack, a non-electric heat pack, a sodium acetate solution heat pack, and an electric heat pack.

19. The method of claim 15 further comprising affixing a liner between the inner strip and the interior surface.

20. An athletic scarf, comprising:
a flat, pliable, substantially rectangular body having a first end, a second end, a top end and a bottom end, and further having a facing surface and an opposing interior surface, bounded by the first and second ends and top and bottom edges;
an inner strip fastened to the interior surface to form a strip of material having a smaller area than the interior surface and running coextensively with along the length of the interior surface, wherein the inner strip includes at least one means for installing at least one removable heat pack;
means for installing at least one removable heat pack within the inner strip;
first fastening means attached to the interior surface proximate to its first end;
a liner attached between the inner strip and the interior surface;
a pocket accessible through a pocket zipper on the facing surface; and
second fastening means attached to the facing surface of the rectangular body proximate to a second end at a side opposite its first end, wherein the facing surface includes means for attaching an insignia patch, wherein the first and second fastening means comprise joinable means on opposing faces for fastening the athletic scarf about the wearer's neck, wherein the first fastening means is joinable at a plurality of locations along the second fastening means so as to vary the circumference of the athletic scarf to closely match the circumference of the wearer's neck.
